(19) United States
(12) Patent Application Publication
(10) Pub. No.: US 2022/0273638 A1
(43) Pub. Date: Sep. 1, 2022

(54) DESLORATADINE AND/OR LORATADINE FOR TREATMENT AND/OR PROPHYLACTIC TREATMENT OF MELANOMA

(71) Applicant: Belina Pharma AB, Lund (SE)

(72) Inventor: Håkan Olsson, Lund (SE)

(21) Appl. No.: 17/637,449

(22) PCT Filed: Aug. 21, 2020
(86) PCT No.: PCT/SE2020/050805

§ 371 Date: Feb. 22, 2022

(30) Foreign Application Priority Data
Aug. 23, 2019 (SE) 1950968-6

(51) Int. Cl. A61K 31/4545; A61P 35/00

(52) U.S. Cl. CPC A61K 31/4545; A61P 35/00

(57) ABSTRACT

Disclosed herein is a pharmaceutical composition comprising a H1 antihistamine being desloratadine and/or loratadine, for use in the treatment and/or prophylactic treatment of melanoma.

### FIELD OF THE INVENTION

This invention pertains in general to the field of melanoma. More particularly the invention relates to a medicament for treatment and prophylactic treatment of melanoma.

### BACKGROUND OF THE INVENTION

It is known that Melanoma, also known as malignant melanoma, is a type of cancer that develops from the pigment-containing cells known as melanocytes. Melanomas typically occur in the skin.

Using sunscreen and avoiding UV light may prevent melanoma. Treatment is typically removal by surgery. For those in whom melanoma has spread, for instance to nearby lymph nodes, immunotherapy, biologic therapy, radiation therapy, or chemotherapy may improve survival.

Melanoma is the most dangerous type of skin cancer. Globally, in 2012, it newly occurred in 232,000 people. In 2015 there were 3.1 million with active disease which resulted in 59,800 deaths. Melanoma is the fifth most common malignancy in Sweden, and its incidence has more than doubled in the last 25 years.

Melanomas may develop from a mole with changes such as an increase in size, irregular edges, change in color, itchiness, or skin breakdown. As such, it is sometimes hard for an individual to correctly diagnose melanoma.

As such, there is a need for new treatments and preventive measures melanoma.

### SUMMARY OF THE INVENTION

It is an object of the present invention, considering the disadvantages mentioned above, to provide pharmaceutical composition for use in the treatment and/or prophylactic treatment of melanoma, wherein the composition comprises desloratadine and/or loratadine.

### BRIEF DESCRIPTION OF THE DRAWINGS

These and other aspects, features and advantages of which the invention is capable of will be apparent and elucidated from the following description of embodiments of the present invention, reference being made to the accompanying drawings, in which

FIG. 1 is a plot showing melanoma specific survival in patients taking and not taking desloratadin,

FIG. 2 is a plot presenting the melanoma specific survival in patients taking and not taking desloratadin in patients under 65 and above 65 years at diagnosis,

FIG. 3 is a plot presenting the overall survival in patients taking and not taking desloratadin in patients under 65 and above 65 years at diagnosis, and

FIG. 4 is a plot presenting the chance of no new primary melanoma after first melanoma diagnosis.

### DESCRIPTION OF EMBODIMENTS

The following description focuses on an embodiment of the present invention applicable to a pharmaceutical composition for use in the treatment and prophylactic treatment of melanoma.

Chronic inflammation resulting in cancer has been known for more than a century and a half, but only relatively recently have we started to apply that knowledge to find and develop anti-inflammatory agents that can be used in cancer therapy. Anti-inflammatory allergy medications present a particularly good starting place for such as search, and H2-antihistamines such as cimetidine have been extensively shown to have promise as adjuvant drugs to treat multiple cancers, but until recently, modern H1-antihistamines have not been studied in relation to cancer therapy despite being ubiquitous, affordable, and safe, as well as—crucially—having much lower toxicity than conventional chemotherapeutics.

Database mining has proven a very powerful tool for finding unseen trends in treatment results. The Swedish Drug Register contains data on pharmaceuticals, supplies, and food taken prescription or equivalent in pharmacy from 2005 onwards. The number of prescriptions is almost 100 million a year. The register is updated with new information every month. The Swedish Cancer Registry was founded in 1958 and covers the entire population of Sweden. Approximately 50 000 malignant cases of cancer is registered every year in Sweden. It is compulsory for every health care provider to report newly detected cancer cases to the registry. A report has to be sent for every cancer case diagnosed at clinical-, morphological-, other laboratory examinations as well as cases diagnosed at autopsy. Thus, these registers provide a very good foundation for database mining seen from an international standard.

We investigated the effects of the six major H1-antihistamines (cetirizine, clemastine, desloratadine, ebastine, fexofenadine and loratadine) used in Sweden on melanoma-specific and overall mortality in a nation-wide register-based study of all 35,905 Swedish individuals with newly diagnosed melanoma between 2005 and 2014. Both peri- and post-diagnostic antihistamine use was analyzed, using Cox regression models, as well as different subgroups of patients with regard to age, gender (as a proxy for sex) and tumor status.

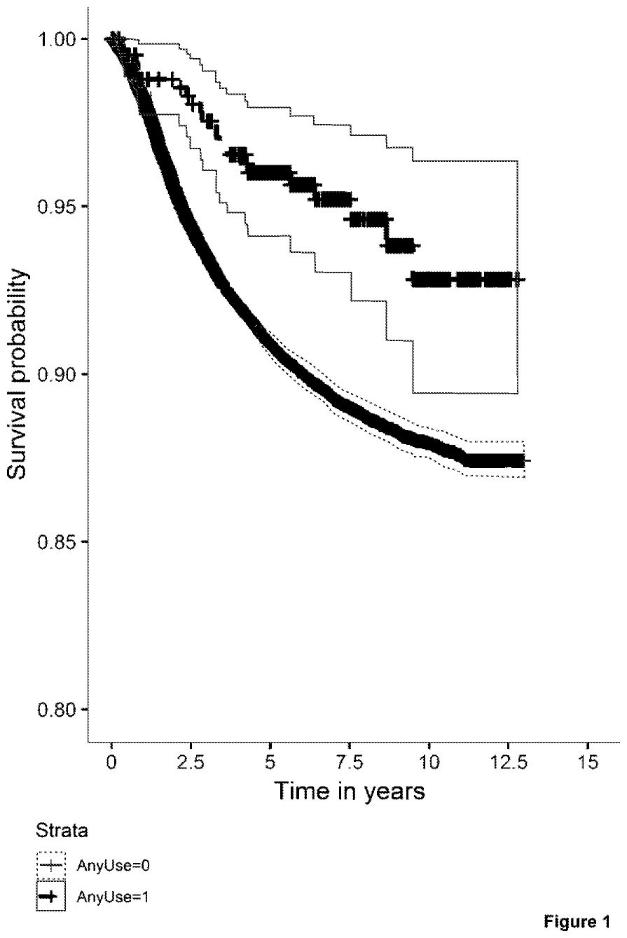

The results are robust, as the study was done on the entire Swedish population. The study had access to more tumor data than what is present in the Swedish Cancer Register, allowing us to adjust our analyses based on Breslow as well as Clark measures. 35,905 individuals in Sweden were newly diagnosed with invasive melanoma of the skin 2005 — 2014. The most common antihistamines used for this patient group (cetirizine, clemastine, desloratadine, ebastine, fexofenadine and loratadine) are summarized in Table 1. The survival effects of the different antihistamines are shown in FIG. 1 (Kaplan-Meier).

It was surprisingly found that different antihistamines had different effects on survival, which suggests that while the mechanism of action may in part be histamine receptor 1-dependent, it is not solely so, rather, some other mechanisms of action may be involved. It is however hypothesized that it can be concluded that the effects are not due to some underlying confounding diagnosis, such as allergic rhinitis, as the effects should then have been more uniform for the different antihistamines in direction. Furthermore, a study undertaken by Hemminki et al. on medically diagnosed allergic rhinitis and cancer risk found a somewhat increased risk of melanoma for those with allergies.

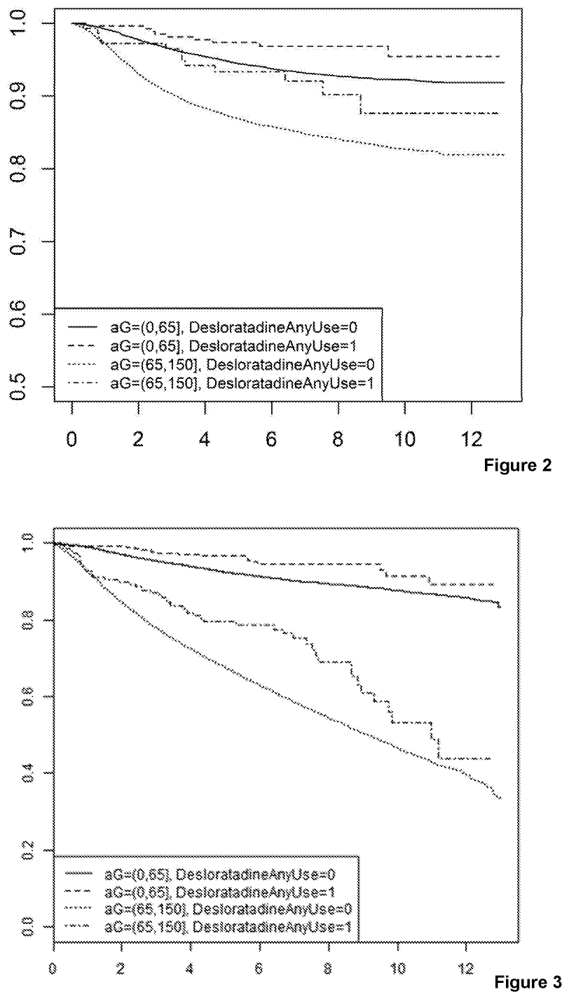

As is summarized in FIGS. 1 to 4, a consistently beneficial effect of desloratadine and loratadine use on melanoma survival was found, regardless of age, gender and tumor status. Also found was a consistently negative effect on melanoma survival of clemastine use. Thus, when conduction a national population based study relating melanoma patients, the present inventors found the surprising result that treatment using desloratadine and/or loratadine has a positive effect on treatment and prophylactic treatment of melanoma.

In one embodiment is provided a pharmaceutical composition for use in the treatment and/or prophylactic treatment of melanoma, wherein the composition comprises desloratadine and/or loratadine.

Table 5 show survival advantages for desloratadine and loratadine (very low hazard ratios of dying from melanoma).

Both melanoma-specific mortality and overall mortality was included in the analyses, as it was hypothesize that the true melanoma-specific mortality is in fact underestimated in the Swedish Cause of Death Register, as autopsies are not routinely performed in Sweden.

Prescription free use precludes us from ascertaining the full effect of some of the antihistamines, particularly cetirizine, ebastine and loratadine, as these were all available without a prescription in Sweden throughout the study period, desloratadine and clemastine were not available without a prescription until 2014, and fexofenadine was first made available for prescription free purchase in 2011, so a full/nearly full effect can be appreciated for these drugs.

While it would have been preferable to have a full background on all patients, including socioeconomic data, UV exposure and genetic mutations, it is not expected that the findings are skewed along any socioeconomic gradient or unknown biological factor. Having done multiple subgroup analyses based on the available variables, we same results are found in each, which strongly suggests that the effects of desloratadine and loratadine on melanoma mortality is dependent on true properties of those drugs on major mechanisms crucial to cancer cells. The study also demonstrated that desloratadine and loratadine use improve survival independent of age, gender, tumour thickness and lymph gland metastases.

In one embodiment, the melanoma cancer is cutaneous melanoma and/or uveal melanoma.

In one embodiment, the melanoma cancer is cutaneous melanoma.

In one embodiment, the cutaneous melanoma is selected from superficial spreading, nodular, lentigo maligna and acral lentiginous melanoma.

The data also indicated that longer therapy is better than shorter.

In one embodiment, the treatment period is at least 50 days, such as at least 100 days, such as at least 200 days, such as at least 1 year, such as at least 3 years.

In one embodiment, the treatment is peridiagnostic, such as from at least 100 days, such at least 3 months, such as at least 6 months, such as at least 1 year before, and such as from at least 100 days, such at least 3 months, such as at least 6 months, such as at least 1 year, after melanoma diagnosis.

The improvement seen for melanoma patients using the H1 receptor antihistamines being loratadine or desloratadine include improved prognosis as well as increase in survival rate.

Thus, in one embodiment of the present invention, the treatment seeks to improve the prognosis for a patient diagnosed with melanoma. In one embodiment, the treatment is a method of improving the prognosis for a patient diagnosed with melanoma.

In one embodiment of the present invention, the treatment seeks to increase the survival time for a patient diagnosed with melanoma. In one embodiment, the treatment is a method to increase the survival time for a patient diagnosed with melanoma.

The study demonstrates that the treatment can be a co-treatment with other melanoma treatments.

In one embodiment, the treatment is a co-treatment of a patient diagnosed with melanoma. In one further embodiment, said co-treatment is selected from treatment with radiotherapy, chemotherapy, and hormonal treatment.

Desloratadine and loratadine are second-generation H1-antihistamines. Second-generation H1-antihistamines are newer drugs that are more selective for peripheral H1 receptors as opposed to the central nervous system H1 receptors and cholinergic receptors. They are very polar compounds, which mean that they do not cross the blood-brain-barrier (BBB) and will act mainly outside the central nervous system. Also, desloratadine and loratadine have minimal side effects. They remain continuously used and are prescribed to adults and children alike, and are widely available without a prescription, due to their safety. The fact that desloratadine and loratadine are both safe and commonplace drugs make them excellent candidates for repurposing purposes.

In this study, the drug usage is estimated based on drug prescriptions. This information reflects usage fairly accurate, especially for persons with multiple prescriptions. A confounding factor is the possibility of off the counter purchase of cetirizine and loratadine, though this should only dilute any seen effects. Desloratadine and clemastine however, were both prescription drugs, during the time of this study.

Thus, in an embodiment of the present invention, the daily dose of desloratadine and/or loratadine corresponds to the defined daily dose (DDD).

The DDD is a statistical measure of drug consumption, defined by the World Health Organization (WHO).

Five milligram of desloratadine is the commonly prescribed daily dose in the prescription register and very few patients have been given a higher daily dose. As such, this study shows a clear benefit from antihistamine treatment in normal dose regimes, but other dose regimes are equally possible, for instance a higher dose for perioperational treatment.

Thus, in one embodiment of the present invention, the dose of the desloratadine is between 2.5 to 45 mg per day, preferably between 5 to 20 mg per day, most preferably 5 mg per day.

Multi dose studies with 45 mg per day of desloratadine did not show any further clinical side effects (FASS, Swedish Medicines Compendium for healthcare professionals), showing he possibility for high doses of desloratadine.

Thus, in one embodiment, the dose of the desloratadine is between 45 to 250 mg per day, preferably between 85 to 150 mg per day most preferably 100 mg per day.

A recommended dose for loratadine is 10 mg/day for a person over 6 years of age and 30 kg body weight. Overdoses of loratadine have been shown to increase the incidence of anticholinergic symptoms—Somnolence, tachycardia and headache have been reported in connection with overdoses (FASS, Swedish Medicines Compendium for healthcare professionals).

In one embodiment of the present invention, the dose of the loratadine is between 2.5 to 50 mg per day, preferably between 5 to 25 mg per day, most preferably 10 mg per day.

According to an embodiment, the treatment and/or prophylactic treatment is for patients with high risk factors for or developing a new melanoma, or having been treated for melanoma, with high risk for having a recurrence of melanoma.

In the context of the present specification, the term “therapy” and “treatment” includes prevention or prophylaxis, unless there are specific indications to the contrary. The terms “therapeutic” and “therapeutically” should be construed accordingly.

According to an embodiment, treatment does also encompass pre-treatment, i.e. prophylactic treatment.

According to an embodiment, the term “prophylaxis” or “prophylactic” includes primary prophylaxis, secondary prophylaxis, tertiary prophylaxis or periodic prophylaxis, unless there are specific indications to the contrary. Primary prophylaxis refers to the preventive treatment of an initial disease. Secondary prophylaxis refers to reducing the incidence of recurrence or reactivation of a pre-existing disease. Teritary prophylaxis refers to continuous treatment started after the onset of a disease to mitigate further damage. Periodic prophylaxis refers to periodic prophylactic treatment given for shorter periods of time. For instance, a H1 receptor antihistamine being desloratadine or loratadine used in perioperational treatment may be used a secondary prophylaxis for reducing the incidence of recurrence or reactivation of a pre-existing melanoma cancer.

Thus, in one embodiment of the present invention, treatment does relate to primary prophylaxis, secondary prophylaxis, tertiary prophylaxis or periodic prophylaxis.

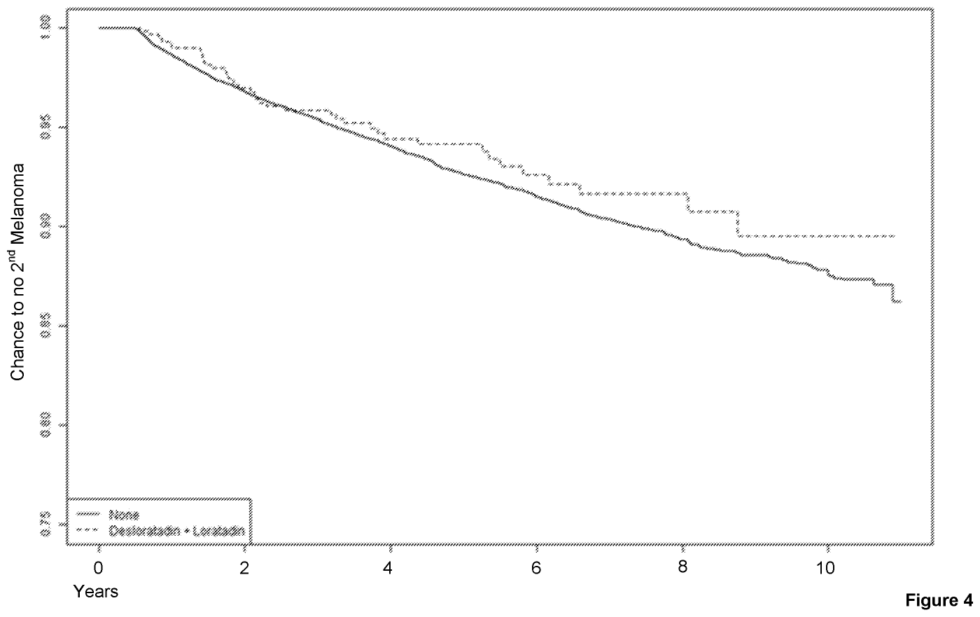

As can be seen in FIG. 4, the prophylactic effect from desloratadine and loratadine was further studied through the risk of developing a second melanoma after the first one. It was found that Desloratadine and Loratadine reduces significantly the risk of a new primary melanoma when use is longer than 3 years after the first diagnosis (HR 0.88, p<0.05). This clearly shows the advantage of desloratadine or loratadine and use after the onset of melanoma, for lowering the risk of recurrence.

In one embodiment, the treatment is a method to reduce occurrence of a new primary melanoma for a patient previously diagnosed with melanoma. In one further embodiment, the treatment period is at least 3 years.

Other risk factor exist which raise the risk of developing melanoma, such as carriers of gene mutations or conditions.

In one embodiment, the treatment is prophylactic treatment, and wherein the subject to be prophylactically treated is a subject being a carrier of a CDKN2A, CDK4, P53, and MITF mutation related to melanoma, or carries an inherited condition selected from xeroderma pigmentosum, retinoblastoma, Li-Fraumeni syndrome, Werner syndrome, and certain hereditary breast and ovarian cancer syndromes or combinations thereof

Environmental risk factors may also raise the risk of developing melanoma, such as carriers of gene mutations or conditions.

In one embodiment, the treatment is prophylactic treatment, and wherein subject to be prophylactically treated is a subject with known risk factor(s) for developing melanoma, the risk factor(s) being selected from the group consisting of sun exposure (exposure to ultraviolet (UV) radiation), indoor tanning, moles (many moles or unusual moles called dysplastic nevi or atypical moles), fair skin, family history of melanoma, familial melanoma, inherited conditions increasing risk of melanoma, history of previous skin cancer, race or ethnicity, old age (the median age at which people are diagnosed with melanoma is just above 50 years old) and a weakened or suppressed immune system, and combinations thereof.

A H1 receptor antihistamine, being desloratadine or loratadine, may obviously also be used in method for treating such diseases and disorders as have been disclosed herein e.g. treatment of melanoma. Such a method includes the step of administering an effective amount of the H1 receptor antihistamine being desloratadine or loratadine to a subject in need for such treatment.

Evidently, a H1 receptor antihistamine being desloratadine or loratadine may also be used for the manufacture of a medicament for use in such treatment as disclosed herein, e.g. treatment of melanoma.

Thus, one embodiment relates to a pharmaceutical composition, comprising desloratadine and/or loratadine and an excipient, for use in the treatment and/or prophylactic treatment of melanoma.

An excipient is a natural or synthetic substance formulated alongside the active ingredient of a medication. Purpose includes stabilization, bulking, facilitating drug absorption, reducing viscosity, or enhancing solubility. Excipients may also be useful for the manufacturing process, to aid in the handling of the active substance. The selection of appropriate excipients also depends upon the route of administration and the dosage form, as well as the active ingredient and other factors. In one aspect of the invention, the pharmaceutical composition comprises an excipient selected from the list containing lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, methyl cellulose, polyethylene glycol, poloxamers, lactose, hydroxypropylmethylcellulose (HPMC), hydroxypropyl cellulose (HPC), lactose, and polyvinylpyrrolidone. In one further aspect of the invention, the S-ARI is administered orally.

Material and Methods

We investigated the effects of the six major H1-antihistamines (cetirizine, clemastine, desloratadine, ebastine, fexofenadine and loratadine) used in Sweden on melanoma-specific and overall mortality in a nation-wide register-based study of all 35,905 Swedish individuals with newly diagnosed melanoma between 2005 and 2014. Both peri- and post-diagnostic antihistamine use was analyzed, using Cox regression models, as well as different subgroups of patients with regard to age, gender (as a proxy for sex) and tumor status.

Our study population included all of the 35,905 individuals with newly diagnosed melanoma from 2005 through 2014, identified using the Swedish National Quality Register for Malignant Skin Melanoma (hereafter referred to as the Melanoma Register), a database comprising all cases of malignant melanoma of the skin in Sweden since 1990. Melanoma in situ is not included in this register, nor melanoma in any other site than the skin. It has a coverage of approximately 98%, and in addition to variables such as age and TNM status, the Melanoma Register also includes information on the histopathology of the tumors, such as Breslow and Clark measures. Use of the six major antihistamines was established using the Swedish Prescribed Drug Register, a record of all dispensed prescribed pharmaceuticals in Sweden since July 1st 2005. To be included in the registry at least 2 weeks of prescription is required. Prescription free antihistamine use, non-dispensed doses, or use of other, less common, types of antihistamines is here considered non-use. Causes of death were obtained from the Swedish Cause of Death Register, a database where all deaths have been recorded since 1952. Data was pseudo-anonymized and the study was approved by the Regional Ethics Board.

We analyzed both the peri- and cumulative post-diagnostic use of antihistamines among melanoma patients. Peri-diagnostic use was a proxy for ever-use, and is here defined as use within six months pre-diagnosis and six months post-diagnosis, where the main antihistamine (in terms of dispensed defined daily doses, or DDDs) used by each individual during this time was identified. The analysis was done using a Cox regression model with time to even or censoring in years as a time scale. Subgroup analyses were made for groups based on age and gender (as a proxy for sex).

For the cumulative post-diagnostic antihistamine use analysis, mortality rates for antihistamine users were compared to those of non-users through a Cox regression model allowing for time varying covariates, and survival time was measured in years since diagnosis. This allowed for a single individual to provide data as both a non-user and user of different antihistamines. Also, to further study the prophylactic effect from desloratadine and loratadine, the risk of developing a second melanoma after the first one was studied.

The impact of the cumulative DDDs was assessed after log transformation of the DDDs plus one3. Cox regression analyses were stratified for patient age at diagnosis, tumor size and node status (T and N status, respectively). A lag-time analysis was performed as described by Tamim et al. and Richardson et al. Statistical analyses were performed using R version XXX6. Two-tailed P-values were used, and a test based on Schoenfeld residuals was done to assess the proportional hazards assumption, and was accepted, both for melanoma-specific and overall mortality.

Results

Below are summarized the results from the study. We found a consistently beneficial effect of desloratadine and/or loratadine use on melanoma survival, regardless of age, gender and tumor status (FIGS. 1 to 3), and a consistently negative effect on melanoma survival of clemastine use.

The tables list drugs which are used and the corresponding hazard ratios (HR). A hazard ratio below 1.0 indicates better survival, while as hazard ratio above 1.0 indicates worse survival, when compared to non-users. The confidence interval (95% CI) indicates the level of uncertainty around the measure of effect. A 95% confidence interval means that the confidence interval covers the true value in 95 of 100 studies performed. The p-value is a probability, where a small p-values correspond to strong evidence. P<0.05 indicates a statistically significant difference between groups.

It was found that the melanoma specific mortality for desloratadine was HR (hazard ratio) 0.75 (0.55-1.02), P=0.0683

If one looks at DDD:s, the result is 0.93 (0.87-1.00), P=0.0496

The results are similar both for men and women.

Men <65; HR 0.69 (0.37-1.29), P=0.243

Men >65; HR 0.77 (0.45-1.33), P=0.346

Women <65; HR 0.57 (0.25-1.28), P=0.173

Women >65; HR 0.80 (0.44-1.45), P=0.456

If one adjusts the results for Breslow thickness (a prognostic factor in melanoma of the skin indicating how deeply tumor cells have invaded), tables 1 and 2 show that the results remain consistent (for different thicknesses) for loratadine and desloratadine. The Breslow thickness measures (in millimetres) the distance between the upper layer of the epidermis and the deepest point of tumour penetration. Also shown are comparative results for cetirizine, clemastine, fexofenadine and ebastine, all which show lack of improval of survivability.

TABLE 1
Overall survival adjusted for Breslow thickness

| | | 95% CI | | |
|---|---|---|---|---|
| Drug | HR | Lower | Upper | P-Value |
| Cetirizine | 1 | 0.958 | 1.043 | 0.987 |
| Clemastine | 1.114 | 1.042 | 1.191 | 0.002 |
| Fexofenadine | 1.011 | 0.859 | 1.19 | 0.891 |
| Desloratadine | 0.95 | 0.9 | 1.003 | 0.066 |
| Loratadine | 0.962 | 0.906 | 1.022 | 0.214 |
| Ebastine | 1.117 | 1.008 | 1.238 | 0.035 |

TABLE 2
Melanoma specific survival adjusted for Breslow thickness.

| | | 95% CI | | |
|---|---|---|---|---|
| Drug | HR | Lower | Upper | P-Value |
| Cetirizine | 1.006 | 0.936 | 1.081 | 0.876 |
| Clemastine | 1.027 | 0.902 | 1.169 | 0.689 |
| Fexofenadine | 0.979 | 0.776 | 1.234 | 0.857 |
| Desloratadine | 0.883 | 0.801 | 0.973 | 0.012 |
| Loratadine | 0.858 | 0.754 | 0.976 | 0.019 |
| Ebastine | 1.138 | 0.987 | 1.312 | 0.075 |

Notably, not only is the HR value low for loratadine and desloratadine, the P-values of 0.012 and 0.019, respectively, are extremely low compared to for the other listed drugs.

The same is true if one adjusts the results for Breslow thickness and lymph gland metastases, as can be seen in tables 3 and 4.

TABLE 3
Melanoma specific survival adjusted for Breslow
thickness and lymph gland metastases.

| | | 95% CI | | |
|---|---|---|---|---|
| Drug | HR | Lower | Upper | P-Value |
| Cetirizine | 1.014 | 0.938 | 1.096 | 0.732 |
| Clemastine | 0.993 | 0.853 | 1.155 | 0.924 |
| Fexofenadine | 0.935 | 0.7 | 1.25 | 0.652 |
| Desloratadine | 0.889 | 0.797 | 0.992 | 0.035 |
| Loratadine | 0.851 | 0.737 | 0.983 | 0.028 |
| Ebastine | 1.03 | 0.834 | 1.272 | 0.785 |
| Bm | 1.047 | 1.044 | 1.05 | 0 |
| SN | 0.903 | 0.811 | 1.006 | 0.063 |

TABLE 4
Overall survival adjusted for Breslow
thickness and lymph gland metastases.

| | | 95% CI | | |
|---|---|---|---|---|
| Drug | HR | Lower | Upper | P-Value |
| Cetirizine | 0.991 | 0.946 | 1.038 | 0.691 |
| Clemastine | 1.101 | 1.022 | 1.187 | 0.011 |
| Fexofenadine | 1.013 | 0.847 | 1.211 | 0.886 |
| Desloratadine | 0.953 | 0.895 | 1.014 | 0.129 |
| Loratadine | 0.964 | 0.903 | 1.03 | 0.276 |
| Ebastine | 1.076 | 0.947 | 1.223 | 0.261 |
| Bm | 1.039 | 1.037 | 1.042 | 0 |
| SN | 1.103 | 1.026 | 1.186 | 0.008 |

In table 5, results for peridiagnostic use (6 months before and after melanoma diagnosis) of antihistamines and melanoma mortality is summarized.

TABLE 5
Peridiagnostic use, melanoma mortality, adjusted for Breslow,
sentinel lymph node status and education level.

| | | 95% CI | | |
|---|---|---|---|---|
| Drug | HR | Lower | Upper | P-Value |
| Clemastine | 1.005 | 0.582 | 1.736 | 0.984 |
| Cetirizine | 0.962 | 0.638 | 1.453 | 0.856 |
| Loratadine | 0.534 | 0.277 | 1.028 | 0.061 |
| Ebastine | 1.032 | 0.332 | 3.204 | 0.957 |
| Fexofenadine | 0.901 | 0.225 | 3.609 | 0.883 |
| Desloratadine | 0.569 | 0.329 | 0.982 | 0.043 |

To further study the prophylactic effect from desloratadine and loratadine, the risk of developing a second melanoma after the first one was studied. It was found that Desloratadine and Loratadine reduces significantly the risk of a new primary melanoma when use is longer than 3 years after the first diagnosis (HR 0.88, p<0.05). This is shown in FIG. 4, where that chance of not developing a new primary melanoma after first melanoma diagnosis adjusted for age and sex. No significant difference was seen if follow up is counted from day 1, for a use at least 3 years after diagnosis of desloratadine or loratadine, users have a significant lower risk of developing a second melanoma (p<0.05).

Although the present invention has been described above with reference to (a) specific embodiment(s), it is not intended to be limited to the specific form set forth herein. Rather, the invention is limited only by the accompanying claims and, other embodiments than the specific above are equally possible within the scope of these appended claims, e.g. different than those described above.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1. A method of treatment and/or prophylactic treatment of melanoma, comprising administering to a subject a pharmaceutical composition comprising desloratadine and/or loratadine.

2. The method according to claim 1, wherein the melanoma is cutaneous melanoma and/or uveal melanoma.

3. The method according to claim 1, wherein a daily dose of desloratadine and/or loratadine corresponds to a defined daily dose (DDD).

4. The method according to claim 1, wherein the daily dose of desloratadine is from 2.5 to 45 mg per day.

5. The method according to claim 1, wherein the daily dose of loratadine is from 2.5 mg to 50 mg.

6. The method according to claim 1, wherein a treatment period is at least 50 days.

7. The method according to claim 1, wherein the treatment is prophylactic treatment, and wherein the subject to be prophylactically treated is a subject being a carrier of a CDKN2A, CDK4, P53, and MITF mutation related to melanoma, or carries an inherited condition selected from xeroderma pigmentosum, retinoblastoma, Li-Fraumeni syndrome, Werner syndrome, and certain hereditary breast and ovarian cancer syndromes or combinations thereof.

8. The method according to claim 1, wherein the treatment is prophylactic treatment, and wherein the subject to be prophylactically treated is a subject with known risk factor(s) for developing melanoma, the risk factor(s) being selected from the group consisting of sun exposure, indoor tanning, moles, fair skin, family history of melanoma, familial melanoma, inherited conditions increasing risk of melanoma, history of previous skin cancer, race or ethnicity, old age and a weakened or suppressed immune system, and combinations thereof.

9. The method according to claim 1, wherein the treatment is a method of improving the prognosis for a patient diagnosed with melanoma.

10. The method according to claim 1, wherein the treatment is a method to increase the survival time for a patient diagnosed with melanoma.

11. The method according to claim 1, wherein the treatment is a method to reduce occurrence of a new primary melanoma for a patient previously diagnosed with melanoma.

12. The method according to claim 1, wherein the daily dose of desloratadine is between 5 to 20 mg per day.

13. The method according to claim 1, wherein the daily dose of desloratadine is 5 mg per day.

14. The method according to claim 1, wherein the daily dose of loratadine is from 5 mg to 25 mg.

15. The method according to claim 1, wherein the daily dose of loratadine is 10 mg.

16. The method according to claim 1, wherein a treatment period is at least 100 days.

17. The method according to claim 1, wherein a treatment period is at least 200 days.

18. The method according to claim 1, wherein a treatment period is at least 1 year.
